(19) United States
(12) Patent Application Publication
(10) Pub. No.: US 2025/0368596 A1
(43) Pub. Date: Dec. 4, 2025

(54) PROCESS FOR THE DECARBOXYLATIVE KETONIZATION OF C2-C8 CARBOXYLIC ACIDS, DERIVATIVES OR MIXTURES THEREOF

(71) Applicant: SPECIALTY OPERATIONS FRANCE, Lyon (FR)

(72) Inventors: Olivier Back, Lyon (FR); Zhen Yan, Taiyuan (CN); Isabelle Storet, Serezin de la Tour (FR)

(73) Assignee: SPECIALTY OPERATIONS FRANCE, Lyon (FR)

(21) Appl. No.: 18/877,956

(22) PCT Filed: Jun. 20, 2022
(86) PCT No.: PCT/CN2022/099858

§ 371 Date: Dec. 20, 2024

(51) Int. Cl. C07C 45/48

(52) U.S. Cl. CPC C07C 45/48

(57) ABSTRACT

The present invention relates to a process for the manufacture of ketones though a decarboxylative ketonization process of C₂-C₈ carboxylic acids, C₂-C₈ carboxylic acid derivatives or mixtures thereof, in the liquid phase. The present invention further relates to ketones susceptible of being produced by the process of the invention.

### DOMAIN OF THE INVENTION

The present invention relates to a process for the manufacture of ketones though a decarboxylative ketonization process of C₂-C₈ carboxylic acids, C₂-C₈ carboxylic acid derivatives or mixtures thereof, in the liquid phase. The present invention further relates to ketones susceptible of being produced by the process of the invention.

### BACKGROUND OF THE INVENTION

Volatile ketones are versatile compounds which can be used in a variety of applications. Acetone (CH₃—CO—CH₃) is for example used directly as a solvent or as a raw material for the manufacture of some green, non-VOC solvents like methylisobutylketone (MIBK), diisobutylketne (DIBK), hexyleneglycol (HGL) or glycerol ketals, in particular 2,2-dimethyldioxolane-4-methanol, also sold under the trademark Augeo®, and used in the F&F industry, for example for the production of raspberry ketone. Ketones having a chain length of eleven carbon atoms up to 18 carbon atoms are particularly suitable in the detergent applications and can be easily derivatized for other targeted applications like for example coating, agrochemistry or Home and Personal Care.

Several processes related to the manufacture of ketones from acid or acid derivatives have been described in the past either in the gas or liquid phase. Compared to a liquid phase process, a gas phase process requires the use of more sophisticated equipment and catalysts, higher temperature and therefore higher operating cost. The productivity of such gas phase process is in addition generally lower than the liquid phase process.

In the liquid phase, patent applications WO 2016/177842, WO 2018/087179 or WO 2018/033607 disclose a process for decarboxylative ketonization of fatty acids that is acids wherein the number of carbon atoms is higher than or equal to 8, and wherein the step of decarboxylation is performed at a temperature strictly above 270° C. and up to 400° C. These processes cannot be easily implemented for the production of ketones from carboxylic acids having a boiling point below the temperature of the process and/or for the production of ketones having a boiling point below the temperature of the process as it requires to operate under pressure with the use of an autoclave while managing the release of the formed CO2 and water. The pressure also has to be monitored and controlled in order to avoid pressure build-up during the process.

The present invention provides a new process for the decarboxylative ketonization of volatile carboxylic acids, in the liquid phase which overcomes the drawbacks of the prior art.

### BRIEF DESCRIPTION

A first aspect of the present invention refers to a process (P) for the decarboxylative ketonization of C₂-C₈ carboxylic acids, C₂-C₈ carboxylic acid derivatives or mixtures thereof into corresponding ketones, in liquid phase, wherein:
a. said carboxylic acids, carboxylic acid derivatives or mixtures thereof, are progressively fed to a composition (C) comprising at least one metal compound or at least an elementary metal as catalyst and a solvent having a boiling point at least 40° C. higher than the process temperature, and wherein
b. the formed ketone is continuously distilled off.

In another aspect the present invention refers to a ketone or a composition of ketones obtainable according to the process (P) of the present invention.

The present invention further refers to a composition comprising at least one ketone having a bio-based carbon content higher than or equal to 75%.

In another aspect, the present invention relates to the use of a ketone or a composition of ketones obtainable according to the process (P) of the present invention or a ketone or a composition of ketones having a bio-based carbon content higher than or equal to 75%, as solvent or as raw material (intermediate) for the preparation of compounds useful in the field of home and personal care, agrochemistry, various industrial processes, coatings, fragrances, veterinary and pharmaceutical applications.

### DETAILED DESCRIPTION

Throughout the description, including the claims, the articles “a”, “an” and “the” are used to refer to one or to more than one (i.e., to at least one) of the grammatical object of the article.

Throughout the description, including the claims, the term “and/or” includes the meanings “and”, “or” and also all the other possible combinations of the elements connected to this term.

Throughout the description, including the claims, the term “comprising one” should be understood as being synonymous with the term “comprising at least one”, unless otherwise specified, and “between” should be understood as being inclusive of the limits. It is specified that, in the continuation of the description, unless otherwise indicated, the values at the limits are included in the ranges of values which are given.

The expression “comprise” should be understood as including equally “consist of” or “consist substantively of”.

It should be noted that in specifying any numerical range, such as a range of concentration, conversion rate or selectivity, any particular upper limit can be associated with any particular lower limit.

If not specified otherwise, a percentage content is on weight basis.

In the present disclosure, the expressions “bio-based material”, “bio-sourced material” or “natural material” designate a product that is composed, in whole or in significant part, of biological products or renewable agricultural materials (including plant, animal, and marine materials) or forestry materials.

In the present disclosure, the expression “bio-based carbon” refers to carbon of renewable origin like agricultural, plant, animal, fungi, microorganisms, marine, or forestry materials living in a natural environment in equilibrium with the atmosphere. The bio-based carbon content is typically evaluated by the means of the carbon-14 dating (also referred to as carbon dating or radiocarbon dating). Furthermore, in the present invention, the “bio-based carbon content” refers to the molar ratio of bio-based carbon to the total carbon of the compound or the product. The bio-based carbon content can preferably be measured by a method consisting in measuring decay process of ¹⁴C (carbon-14), in disintegrations per minute per gram carbon (or dpm/gC), through liquid scintillation counting, preferably according to the Standard Test Method ASTM D6866-16. Said American standard test ASTM D6866 is said to be equivalent to the ISO standard 16620-2. According to said standard ASTM D6866, the testing method may preferably utilize AMS (Accelerator Mass Spectrometry) along with IRMS (Isotope Ratio Mass Spectrometry) techniques to quantify the bio-based content of a given product.

The terms “Home and Personal care” are to be understood as comprising cosmetic formulations (body and hair care formulations) and detergent formulations (laundry, softeners, hard surface cleaning . . . ) or house care formulations (fresheners, air care devices).

A first aspect of the present invention refers to a process (P) for the decarboxylative ketonization of C₂-C₈ carboxylic acids, C₂-C₈ carboxylic acids derivatives or mixtures thereof into corresponding ketones, in liquid phase, wherein:

1a. said carboxylic acids, carboxylic acid derivatives or mixtures thereof, are progressively fed to a composition (C) comprising at least one metal compound or at least an elementary metal as catalyst and a solvent having a boiling point at least 40° C. higher than the process temperature, and wherein
b. the formed ketone is continuously distilled off.

According to the present invention, C₂-C₈ carboxylic acids, C₂-Cg carboxylic acids derivatives or mixtures are used, preferably C₂-C₈ carboxylic acids or mixture of C₂-C₈ carboxylic acids are used in the process (P).

According to the present invention, the C₂-C₈ carboxylic acids refers to a compound comprising from 2 to 8 carbon atoms and comprising at least one carboxylic acid functionality (—COOH), preferably comprising exactly one carboxylic acid functionality. It is further specified that according to the present invention the carbon atom of the carboxylic acid functionality is included in the count of C₂-C₈ carbon atoms. In a preferred embodiment, the carboxylic acid used in the process (P) can comprise 2, 3, 4, 5, 6, 7 or 8 carbon atoms.

The C₂-C₈ chain of the carboxylic acids used in the present invention may be linear or branched, preferably linear, and saturated or unsaturated, preferably saturated. The C₂-C₈ chain of the carboxylic acids used in the present invention may be optionally substituted, provided the resulting compound remains stable at the process temperature. As examples of substitution, we can cite halogens, or ketones.

In a preferred embodiment, the C₂-C₈ carboxylic acid is preferably selected from the group consisting of acetic acid, propanoic acid, butanoic acid, isobutyric acid, pentanoic acid, isovaleric acid, hexanoic acid, isocaproic acid, heptanoic acid, isoenanthic acid, octanoic acid, 2-ethylhexanoic acid and isocaprylic acid and mixtures thereof.

According to the present invention, the C₂-C₈ carboxylic acid is a bio-based C₂-C₈ carboxylic acid, preferably the bio-based carbon content of the C₂-C₈ carboxylic acid is higher than or equal to 75%, preferably higher than or equal to 80%, more preferably higher than or equal to 90%. In general, the bio-based carbon content of the C₂-C₈ carboxylic acid is lower than or equal to 110%, preferably lower than or equal to 105%, more preferably lower than or equal to 102%, still more preferably lower than or equal to 100%.

According to the present invention, the C₂-C₈ carboxylic acid derivatives refer to derivatives of the C₂-C₈ carboxylic acids as described in the present invention. Accordingly the C₂-C₈ carboxylic acid derivatives used in the process of the present invention may be of formula (I) R1—COO—R2 or of formula (II) R1—CO—O—CO—R2 wherein R1 comprises from 1 to 7 carbon atoms, and wherein R2 is a chain comprising from 1 to 8 carbon atoms. R1 and R2 may be linear or branched, preferably linear, and saturated or unsaturated, preferably saturated. R1 and R2 may be optionally substituted, provided the resulting compound remains stable at the process temperature. As examples of substitution, we can cite halogens, or ketones.

In a preferred embodiment, the C₂-C₈ carboxylic acid derivative is preferably selected from the group consisting of acetic acid derivatives, propanoic acid derivatives, isobutyric acid derivatives, butanoic acid derivatives, hexanoic acid derivatives, isocaproic acid derivatives, pentanoic acid derivatives, isovaleric acid derivatives, heptanoic acid derivatives, isoenanthic acid derivatives, isocaprylic acid derivatives, 2-ethylhexanoic acid derivatives and octanoic acid derivatives.

According to the present invention, the C₂-C₈ carboxylic acid derivative is a bio-based C₂-C₈ carboxylic acid derivative, preferably the bio-based carbon content of the C₂-C₈ carboxylic acid derivative is higher than or equal to 75%, preferably higher than or equal to 80%, more preferably higher than or equal to 90%. In general, the bio-based carbon content of the C₂-C₈ carboxylic acid derivative is lower than or equal to 110%, preferably lower than or equal to 105%, more preferably lower than or equal to 102%, still more preferably lower than or equal to 100%.

According to the present invention, the C₂-C₈ carboxylic acid, the C₂-C₈ carboxylic acid derivatives or mixture thereof are progressively fed to a composition (C) comprising at least one metal compound or at least an elementary metal as catalyst and a solvent having a boiling point at least 50° C. higher than the process temperature.

It is understood that when a single C₂-C₈ carboxylic acid is used in the process (P) a unique symmetrical ketone is obtained as the decarboxylative ketonization product. When a mixture of C₂-C₈ carboxylic acids and/or the C₂-C₈ carboxylic acid derivatives (cut of C₂-C₈ carboxylic acids and/or C₂-C₈ carboxylic acid derivatives) is used in the process (P), a statistical mixture of all possible ketones formed by reactions of the different C₂-C₈ carboxylic acids and/or carboxylic acid derivatives is obtained (“composition of ketones”).

According to the present invention, the catalyst is selected from the group consisting of at least one metal compound or at least one elementary metal. Suitable metals for use in the process in accordance with the present invention are selected from the group consisting of Mg, Ca, Al, Ga, In, Ge, Sn, Pb, As, Sb, Bi, Cd, Ce, Zr and transition metals having an atomic number of from 21 to 30. Suitable metal compounds are oxides of the aforementioned metals, naphthenate salts of the aforementioned metals or acetate salts of the aforementioned metals. Magnesium and iron and their oxides, and in particular magnesium oxide and iron powder, are preferred.

In accordance with a preferred embodiment, the catalyst is selected from the group consisting of MgO, Fe powder, CaO, TiO₂, FeO, Fe2O₃, Fe3O₄, MnCO₃, ZrO₂ and CeO₂.

The decarboxylative ketonization reaction may be described as a two-step reaction, wherein in a first step the catalyst reacts with the C₂-C₈ carboxylic acid or C₂-C₈ carboxylic acid derivative to form an intermediate species followed by a second step wherein the intermediate species decomposes into the desired ketone and a metal oxide which can then react in another decarboxylative ketonization cycle. This second step further produces CO₂. Considering the mechanism of the decarboxylative ketonization reaction involving the formation of an enol or enolate, it is understood that at least one of the C₂-C₈ carboxylic acids or derivatives engaged in the decarboxylative ketonization reaction shall possess at least one hydrogen atom attached to the carbon atom adjacent to the carboxylic acid function —COOH or derivative.

In the first step of a decarboxylative ketonization reaction, it is further indicated that a metal carboxylate is formed as an intermediate species by reaction of the catalyst with the C₂-C₈ carboxylic acid or C₂-C₈ carboxylic acid derivative.

If an elementary metal is used as catalyst, said metal reacts with the C₂-C₈ carboxylic acid or C₂-C₈ carboxylic acid derivative to form an intermediate carboxylate salt of the metal with simultaneous formation of hydrogen gas in the case of carboxylic acid. If a metal oxide is used as a catalyst, the formation of the carboxylate salt is accompanied by the simultaneous formation of water. The overall equation for the carboxylate salt formation (for a metal having a valency of 2 as example) can be represented as follows:

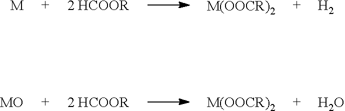

The formation of the intermediate metal carboxylate salt can be conveniently monitored by in situ IR analysis. The carbonyl stretching absorption band of the acid is subject to a bathochromic shift in the metal carboxylate salt which allows the monitoring of the reaction progress.

According to the present invention, the amount of catalyst used in the process (P) is generally lower than or equal to 20 wt % based on the total amount of C₂-C₈ carboxylic acid, C₂-C₈ carboxylic acid derivative or mixture thereof used in the process (P), preferably lower than or equal than 10 wt %, still more preferably lower than or equal to 5 wt %. In general the amount of catalyst used in the process (P) is comprised between 0.01 wt % and 20 wt %, preferably comprise between 0.01 wt % and 10 wt % based on the total amount of C₂-C₈ carboxylic acid, C₂-C₈ carboxylic acid derivative or mixture thereof used in the process (P).

In general, the process (P) is carried out at a temperature (“process temperature”) higher than or equal to 250° C., preferably higher than or equal to 270° C., more preferably higher than or equal to 280° C. In general, the process (P) is carried out at a temperature (“process temperature”) lower than or equal to 400° C., preferably lower than or equal to 370° C., more preferably lower than or equal to 350° C.

In general, the process (P) is carried out at atmospheric pressure or sub-atmospheric pressure.

The decarboxylative ketonization process of the present invention is carried out in the liquid phase in a solvent. The solvent used in the process (P) is selected from the group consisting of solvents having a boiling point at least 40° C. higher than the process temperature, preferably at least 50° C. higher than the process temperature. The solvent is selected for not reacting under the process conditions with the catalyst, the ketone product, the intermediate carboxylate salt, water or the C₂-C₈ carboxylic acids or C₂-C₈ carboxylic acid derivatives and for being thermally stable at high temperature.

According to a specific embodiment, the solvent may be a ketone having a boiling point at least 50° C. higher than the process temperature. According to particular embodiment, the solvent used in the process (P) may be a heat transfer fluid, for example a heat transfer fluid from the Marlotherm®, Fragoltherm®, Therminol® or Dowtherm® series, in particular Marlotherm®SH.

According to particular embodiment, the solvent used in the process (P) may be a ketone, in particular isostearone, laurone, myristone, palmitone, stearone or their mixtures or mixture of ketones obtained from the decarboxylative ketonization reaction conducted on C₁₂-C₁₈ saturated fatty acid mixtures, for example cut of fatty acids obtained from coconut oil, palm kernel oil, tallow oil or palm oil. The preferred solvent used in the process (P) is isostearone.

According to another specific embodiment the solvent may be a ketone obtained by a decarboxylative ketonization process of isostearic acid, caprylic acid, capric acid, lauric acid, myristic acid, palmitic acid, stearic acid, arachidic acid, behenic acid, lignoceric acid, cerotic acid, oleic acid, linoleic acid, linolenic acid, erucic acid, palmitoleic acid or mixtures thereof, preferably isostearic acid.

According to the present invention, the C₂-C₈ carboxylic acid, C₂-C₈ carboxylic acid derivative or mixture thereof are progressively fed to the composition (C). In particular the C₂-C₈ carboxylic acid, C₂-C₈ carboxylic acid derivative or mixture thereof are fed at a rate avoiding the accumulation of the free C₂-C₈ carboxylic acid in the reaction medium.

For example the C₂-C₈ carboxylic acid, C₂-C₈ carboxylic acid derivative or mixture thereof can be progressively fed to the composition (C) over a period of 1 h to 24 hours. Indeed it has been surprisingly observed for the liquid phase ketonization reactions that the presence of residual free carboxylic acid (even at concentration equal or below 50 mol % with respect to the intermediate complex) in the reaction mixture significantly slowed down the decarboxylation kinetic of the intermediate metal complex to the desired ketone. It is therefore critical to control the rate of carboxylic acid or carboxylic acid derivatives (that could generate in-situ free carboxylic acid by hydrolysis) addition such that there is no accumulation of the free carboxylic acid (meaning not complexed).

According to the present invention, the process (P) can lead to the formation of a single ketone or a mixture of ketones. Preferably the ketones formed during the process (P) are selected from the group consisting of acetone (propanone), 2-butanone, 2-pentanone, 3-pentanone, 2-hexanone, 3-hexanone, 2-heptanone, 3-heptanone, 4-heptanone, 2-octaonone, 3-octanone, 4-octanone, 2-nonanone, 3-nonanone, 4-nonanone, 5-nonanone, 3-decanone, 4-decanone, 5-decanone, 4-undecanone, 5-undecanone, 6-undecanone, 5-dodecanone, 6-dodecanone, 6-tridecanone, 7-tridecanone, 7-tetradecanone, 8-pentadecanone.

According to the present invention, the ketone formed during the process (P) is a bio-based ketone, preferably the bio-based carbon content of the ketone is higher than or equal to 75%, preferably higher than or equal to 80%, more preferably higher than or equal to 90%. In general, the bio-based carbon content of the ketone is lower than or equal to 110%, preferably lower than or equal to 105%, more preferably lower than or equal to 102%, still more preferably lower than or equal to 100%.

The formed ketone is continuously distilled off. The formed ketone generally has a boiling point below the process temperature. The formed ketone may be continuously distilled and further condensed, for example by using a distillation or a Dean-Stark apparatus. If the formed ketone has a boiling point above the process temperature, the reaction can be carried out under reduced pressure such that the boiling point of the formed ketone at this reduced pressure is equal or lower than the process temperature in order to facilitate ketone distillation.

Water is continuously formed during the process (P). This water can also be continuously distilled off the reaction and may be condensed together with the formed ketone. In the specific case wherein the formed ketone and the water are not miscible, the formed ketone and water may be easily separated by liquid-liquid or solid-liquid separation. Alternatively, in the event the formed ketone and the water are miscible, for example acetone and water, the formed ketone can be separated from water by any conventional purification method, for example the formed ketone can be distilled from the composition comprising the formed ketone and water, the formed ketone can also be separated from water by membrane purification or pervaporation.

The process (P) of the present invention present several advantages, notably the process avoids autogenous pressure build-up, is compatible with cheap and available catalysts and it avoids the use of costly equipment. The work-up of the reaction is simplified: no separation of the catalyst or the solvent from the formed ketone is required as the formed ketone is continuously distilled from the reaction mixture and therefore directly separated. The process of the present invention may be used in a continuous mode, thereby improving the global productivity of the overall process. Finally catalyst which could lead to deleterious effect to the ketone product in particular chromogenic catalysts, like iron oxides, can advantageously be used without deleterious effect as the formed ketone is continuously distilled off the reaction. This is also the case for catalysts than can catalyze ketone decomposition reactions, for example basic catalysts like CaO and MgO, that could catalyze ketone aldolisation/crotonisation side-reactions at high temperature. Those catalysts can now be used in such process without significant yield loss. Globally it can be said that such process combines the advantages of the liquid phase process (lower temperature, standard equipment, cheap catalysts, high productivity etc . . . ) and the gas phase process (easy catalyst/ketone product separation, continuous process . . . ).

As mentioned previously according to a specific embodiment, the solvent may be a ketone obtained by a decarboxylative ketonization process. In this specific embodiment, the decarboxylative ketonization process (P) of the present invention may comprise a preliminary step (a0) wherein a fatty acid, a fatty acid derivative or mixture thereof undergoes a decarboxylative ketonization process following the process described in WO 2016/177842, WO 2018/087179 or WO 2018/033607. In general this preliminary step is conducted in the presence of a catalyst that is selected from the group consisting of at least one elementary metal or at least one metal compound. Suitable metals for use in the process in accordance with the present invention are selected from the group consisting of Mg, Ca, Al, Ga, In, Ge, Sn, Pb, As, Sb, Bi, Cd, Ce, Zr and transition metals having an atomic number of from 21 to 30. Suitable metal compounds are oxides of the aforementioned metals, naphthenate salts of the aforementioned metals or acetate salts of the aforementioned metals. Magnesium and iron and their oxides, and in particular iron powder, are preferred.

In accordance with a preferred embodiment, the catalyst is selected from the group consisting of MgO, Fe powder, TiO₂, CaO, FeO, Fe₂O₃, Fe3O₄, MnCO₃, ZrO₂, and CeO₂.

Advantageously, the catalyst used in the preliminary step a0 is the same as the catalyst used in the decarboxylative ketonization of the C₂-C₈ carboxylic acid, C₂-C₈ carboxylic acid derivatives or mixture thereof such that no additional catalyst is needed. In this case, the crude mixture obtained after decarboxylative ketonization of fatty acids, fatty acids derivatives or mixture thereof in the step a0, composed of ketone product and catalyst can be used as such for the decarboxylative ketonization of the C₂-C₈ carboxylic acid, C₂-C₈ carboxylic acid derivatives or mixture thereof.

The fatty acid is preferably selected from the group consisting of isostearic acid, caprylic acid, capric acid, lauric acid, myristic acid, palmitic acid, stearic acid, arachidic acid, behenic acid, lignoceric acid, cerotic acid, oleic acid, linoleic acid, linolenic acid, erucic acid, palmitoleic acid or mixtures thereof. The fatty acid derivative is generally selected from the group consisting of isostearic acid derivative, caprylic acid derivative, capric acid derivative, lauric acid derivative, myristic acid derivative, palmitic acid derivative, stearic acid derivative, arachidic acid derivative, behenic acid derivative, lignoceric acid derivative, cerotic acid derivative, oleic acid derivative, linoleic acid derivative, linolenic acid derivative, erucic acid derivative, palmitoleic acid derivative or mixtures thereof.

Step (a0) is carried out at a temperature higher than or equal to 250° C., preferably higher than or equal to 270° C., more preferably higher than or equal to 280° C. In general, the step (a0) is carried out at a temperature lower than or equal to 400° C., preferably lower than or equal to 370° C., more preferably lower than or equal to 350° C.

Step (a0) is generally conducted in the liquid phase. However, step (a0) may also be conducted in the gas phase.

In another aspect the present invention refers to a ketone or a composition of ketones obtainable according to the process (P) of the present invention.

The present invention further refers to a ketone or a composition of ketones having a bio-based carbon content higher than or equal to 75%, preferably higher than or equal to 80%, more preferably higher than or equal to 90%. In general, the bio-based carbon content of the bio-based ketone or of the composition of bio-based ketones of the present invention is lower than or equal to 110%, preferably lower than or equal to 105%, more preferably lower than or equal to 102%, still more preferably lower than or equal to 100%.

In another aspect, the present invention relates to the use of a ketone or a composition of ketones obtainable according to the process (P) of the present invention or a ketone or a composition of ketones having a bio-based carbon content higher than or equal to 75%, as solvent or as raw material (intermediate) for the preparation of compounds useful in the field of home and personal care, agrochemistry, various industrial processes, coatings, fragrances, veterinary and pharmaceutical applications.

### EXAMPLES

### Example 1: Preparation of a High Boiling Solvent (Isostearone) Through a Decarboxylative Ketonization Process (Step a0)

The reaction was carried under argon in a reactor equipped with mechanical stirring. In the reactor were introduced 14.31 g of MgO (0.352 mole, 1 eq) and 99.1 g of isostearic acid (Radiacid™ 0907 grade from Oleon, 0.352 mole of fatty acid, 1 eq.)

The temperature was then raised to 250° C. and stirring was started. During this stage, magnesium carboxylate complex is formed along with the generation of water which is distilled out from the reaction medium. After 40 min reaction time at 250° C., FTIR analysis of the crude showed complete conversion of the starting isostearic acid into the intermediate magnesium bis-carboxylate complex. The temperature of the reaction mass is then raised further to 330° C. and the mixture is allowed to stir at this temperature during 3 h40 in order to allow complete decomposition of the intermediate magnesium complex into the desired isostearone (and CO₂) and MgO which is followed-up thanks to FTIR analysis.

The temperature of the mixture is then lowered to 310° C. A distillation bridge with a short height and connected to a 250 mL collection flask is then mounted on the reactor vessel in order to distill out the formed ketone and water.

### Example 2: Decarboxylative Ketonization of Hexanoic Acid (Invention)

In a reactor was introduced the MgO/isostearone suspension recovered as the high boiling point bottom according to Example 1. Under light vacuum (380 mbar) and under stirring was added hexanoic acid (0.352 moles) at a flow rate of 0.37 mL/min.

Water which is formed during hexanoic acid reaction with MgO is distilled out from the reaction medium along with 6-undecanone.

At the end of the hexanoic acid addition, the temperature of the mixture is raised to 315° C. for digestion during which intermediate complex decomposition to the C11 ketone and product distillation is observed (along with CO₂ release).

After 2 h45 of digestion at 315° C. during which the reaction is followed up by FTIR analysis confirming magnesium complex consumption, two other cycles of 1 eq. of hexanoic acid addition (2 h00) followed by 2h45 digestion are carried out (2 additional equivalents of hexanoic acid).

Then a fourth and last cycle is conducted during which the final distillation is maintained at 315° C. for 3 h45 in order to complete the decarboxylation of the complex to the C11-ketone, which is verified by FTIR analysis.

The distillates from the 4 runs are combined, decanted and the top layer organic phase is separated from the aqueous phase.

The liquid is introduced into a 1L reactor and dried under vacuum at 60° C. for 6 hours to remove traces of water.

After vacuum drying, a clear yellow oil was obtained corresponding to an isolated yield of 89% with a purity of 98 mol % (NMR).

1H NMR (CDCl_{3, 400} MHz) δ (ppm): 2.31 (t, J=7.6 Hz, 4H), 1.54-1.46 (m, 4 H), 1.28-1.16 (m, 8H), 0.83 (t, J=6.8 Hz, 6H).

13C NMR (CDCl_{3, 101} MHz) δ (ppm): 211.75, 42.90, 31.6, 23.70, 22.60, 14.02 (terminal CH₃).

### Example 3: Decarboxylative Ketonization of Octanoic Acid (Invention)

The reaction is conducted under an inert argon and the reactor is equipped with a mechanical stirrer a distillation bridge connected to a collector, a heating mattress and a temperature probe.

In the reactor was introduced the MgO/isostearone suspension recovered as the high boiling point bottom according to example 1.

The suspension comprises:
500 g of isostearone (1 mole, 0.5 eq.)
81 g of MgO (2 moles, 1 eq.).

287.5 g of octanoic acid (310.1 mL, 2 moles, 1 eq.) are added into a 500 mL flask which is then inerted under an argon atmosphere. The flask is connected to the reactor through a pump thanks to a stainless steel pipe. The reaction mixture is then heated at 315° C. and stirred.

Octanoic acid is then progressively fed into the reactor corresponding to the addition of 1 equivalent of octanoic acid during 3 h15.

During the octanoic acid addition, the temperature of the reaction mass is maintained at 315° C. and water is progressively distilled off the reactor.

At the end of the octanoic acid addition, the temperature of the reaction mixture is increased at 330° C. during 30 min as the digestion stage in order to complete the intermediate magnesium complex decarboxylation to the ketone product, MgO and CO₂.

At this stage, the amount of recovered C15-ketone is lower than the theoretical amount, therefor a light vacuum (700 mbar) is progressively applied to the reaction vessel in order to facilitate 6-pentadecanone distillation and to recover additional amount of ketone during 25 min.

The pressure is then increased back to 1 atmosphere and a second octanoic acid addition (287.5 g, 310.1 mL, 2 moles, 1 eq.) is performed at a faster addition rate (2 h30 addition this time) and at 330° C. During this second addition, the temperature of the reaction mixture is stable and maintained at 330° C. and water as well as the C15 ketone product are smoothly distilled out from the reactor.

At the end of the second octanoic acid addition, the reaction mixture is maintained at 330° C. during 15 minutes (digestion phase) in order to complete the intermediate complex ketonization to C15 ketone, CO₂ and MgO.

Finally a third and last octanoic acid addition (330.5 g, 356.5 mL, 2.29 moles, 1.15 eq) with an addition time of 3 h45 is performed at 315°. During this third addition, the temperature of the reaction mixture is stable and maintained at 315° C. and water as well as the C15 ketone product are very smoothly distilled out from the reactor.

At the end of this last octanoic acid addition, the temperature of the reaction mass is increased gradually to 330° C. in order to complete the intermediate complex ketonization to C15 ketone, CO₂ and MgO and until no more organic product distillates out from the reactor. In order to recover all the C15 ketone remaining in the reactor, the reaction vessel pressure is decreased down to 900 mbar and a strong release of white vapor is observed for a few seconds. The total digestion time after the third carboxylic acid addition is 1 h40.

Before each carboxylic acid addition cycle, FTIR analysis on the reaction medium is performed to verify the absence of intermediate magnesium carboxylate complex and the organic phase of the distillate is analyzed by 1H NMR to measure the amount of free fatty acid in the distillate. The distillate collection vessel is also drained before each new carboxylic acid addition and the mass of collected water and C15 ketone is determined for each addition cycle after decantation at 50° C.

After vacuum drying, the C15 ketone was obtained corresponding to an isolated yield of 76% with a purity of 98.7 wt %. (NMR).

### Example 4: Decarboxylative Ketonization of Acetic Acid (Invention)

60 g of Marlotherm SH and 2 g of catalysts as shown in Table 1 were loaded into a 100 mL Parr reactor. After closing the reactor, a 20 mL/min nitrogen flow was used to flush the reactor. Then the reactor was heated to the reaction temperature under N2 flow according to Table 1 and stirring. Acetic acid was then fed to the reactor using a syringe pump, with or without a nitrogen flow. The outlet of the reactor was connected to a cold bath to collect products. The reaction was conducted for two to three hours.

The products were analyzed with GC using an internal standard. Results are shown in Table 1.

TABLE 1

| | Rate of addition | Flow rate | Reaction | |
|---|---|---|---|---|
| | of acetic acid | of N₂ | temperature | Yield |
| Catalyst | (mL/min) | (mL/min) | (° C.) | (%) |
| MnCO₃ | 0.1 | 10 | 340 | 67 |
| MnCO₃ | 0.05 | 5 | 340 | 72 |
| TiO₂ | 0.05 | 5 | 340 | 60 |
| MgO | 0.05 | 5 | 340 | 71 |
| Fe₃O₄ | 0.05 | 5 | 340 | 68 |
| CeO₂ | 0.05 | 5 | 340 | 83 |
| CeO₂ (4 g | 0.05 | 0 | 340 | 89 |
| of catalyst | | | | |
| instead of 2 g) | | | | |
| CeO₂ (4 g | 0.05 | 0 | 320 | 84 |
| of catalyst | | | | |
| instead of 2 g) | | | | |
| CeO₂ (4 g | 0.05 | 0 | 300 | 76 |
| of catalyst | | | | |
| instead of 2 g) | | | | |

1. A process (P) for the decarboxylative ketonization of C₂-C₈ carboxylic acids, C₂-Cs carboxylic acid derivatives or mixtures thereof into corresponding ketones, in liquid phase, wherein:
a. the C₂-C₈carboxylic acids, C₂-C₈ carboxylic acid derivatives or mixtures thereof, are progressively fed to a composition C comprising at least one metal compound or at least an elementary metal as catalyst and a solvent having a boiling point at least 40° C. higher than the process (P) temperature, and wherein
b. the formed ketone is continuously distilled off.

2. The process (P) according to claim 1 wherein the C₂-C₈ carboxylic acid, the C₂-Cs carboxylic acid derivatives or mixtures thereof are selected from the group consisting of acetic acid, propanoic acid, butanoic acid, isobutyric acid, pentanoic acid, isovaleric acid, hexanoic acid, isocaproic acid, heptanoic acid, isoenanthic acid, octanoic acid, 2-ethylhexanoic acid and isocaprylic acid, an acetic acid derivative, a propanoic acid derivative, an isobutyric acid derivative, a butanoic acid derivative, a hexanoic acid derivative, an isocaproic acid derivative, a pentanoic acid derivative, an isovaleric acid derivative, a heptanoic acid derivative, an isoenanthic acid derivative, an isocaprylic acid derivative, a 2-ethylhexanoic acid derivative and an octanoic acid derivative, or mixtures thereof.

3. The process (P) according to any one of claim 1, wherein the at least one metal compound or the at least an elementary metal comprises a metal selected from the group consisting of Mg, Ca, Al, Ga, In, Ge, Sn, Pb, As, Sb, Bi, Cd, Zr, Ce and transition metals having an atomic number of from 21 to 30.

4. The process (P) according to claim 1, wherein the process (P) is carried out at a temperature higher than or equal to 250° C.

5. The process (P) according to 1, wherein water is continuously formed during the process (P) and continuously distilled off the reaction.

6. The process (P) according to claim 1, wherein the C₂-C₈ carboxylic acids, C₂-C₈ carboxylic acid derivatives or mixtures thereof are fed at a rate avoiding accumulation of the free C₂-C₈ carboxylic acid.

7. The process (P) according to claim 1, wherein the solvent is a ketone having a boiling point at least 40° C. higher than the process (P) temperature or a heat transfer fluid.

8. The process (P) according to claim 1, wherein the solvent is a ketone obtained by a decarboxylative ketonization process.

9. The process (P) according to claim 1, wherein the process (P) comprises a preliminary step (a0) wherein a fatty acid, a fatty acid derivative or mixture thereof undergoes a decarboxylative ketonization process, preferably the fatty acid is selected from the group consisting of isostearic acid, caprylic acid, capric acid, lauric acid, myristic acid, palmitic acid, stearic acid, arachidic acid, behenic acid, lignoceric acid, cerotic acid, oleic acid, linoleic acid, linolenic acid, erucic acid, palmitoleic acid or mixtures thereof, the fatty acid derivative is selected from the group consisting of isostearic acid derivative, caprylic acid derivative, capric acid derivative, lauric acid derivative, myristic acid derivative, palmitic acid derivative, stearic acid derivative, arachidic acid derivative, behenic acid derivative, lignoceric acid derivative, cerotic acid derivative, oleic acid derivative, linoleic acid derivative, linolenic acid derivative, erucic acid derivative, palmitoleic acid derivative or mixtures thereof.

10. A ketone or a composition of ketones obtained according to the process (P) as defined in claim 1.

11. The ketone or a composition of ketones according to claim 10, wherein the ketone or composition of ketones has a bio-based carbon content higher than or equal to 75%.

12. (canceled)
